(19) United States
(12) Patent Application Publication
(10) Pub. No.: US 2019/0042844 A1
(43) Pub. Date: Feb. 7, 2019

(54) INTELLIGENT VISUAL PROSTHESIS

(71) Applicant: Brown University, Providence, RI (US)

(72) Inventors: Michael PARADISO, Providence, RI (US); Morgan Bruce DeWitt TALBOT, Spokane, WA (US)

(21) Appl. No.: 16/054,547

(22) Filed: Aug. 3, 2018

Related U.S. Application Data
(60) Provisional application No. 62/540,783, filed on Aug. 3, 2017.

(51) Int. Cl. G06K 9/00; G10L 15/22; H04N 7/18; H04R 5/033; H04S 7/00; G08B 21/02; A61F 9/08

(52) U.S. Cl. CPC G06K 9/00671; G10L 15/22; G06K 9/00355; H04N 7/185; H04S 2400/11; H04S 7/304; G08B 21/02; A61F 9/08; G10L 2015/223; H04R 5/033

(57) ABSTRACT

A visual prosthetic system includes a computer system, and a wearable spectacle, the wearable spectacle linked to the computer system and comprising a pair of headphones, a microphone, a depth camera, a sensor, a fish-eye camera and 3D spectacle frame, the computer system configured to receive outputs from the depth camera, the sensor and the fish-eye camera to track a user's hand and a target object simultaneously.

### CROSS REFERENCE TO RELATED APPLICATIONS

This application claims benefit from U.S. Provisional Patent Application Ser. No. 62/540,783, filed Aug. 3, 2017, which is incorporated by reference in its entirety.

### STATEMENT REGARDING GOVERNMENT INTEREST

None.

### BACKGROUND OF THE INVENTION

The invention generally relates to prosthesis devices, and more specifically to intelligent vision prostheses.

There are roughly 32 million blind people worldwide. In the United States there are presently over 1 million blind people and this number is expected to increase to about 4 million by 2050. Surveys have repeatedly shown that Americans consider blindness to be one of the worst possible health outcomes along with cancer and Alzheimer's disease. The prevalence and concern about blindness stand in sharp contrast to our ability to ameliorate it.

One method used to ameliorate it is referred to as visual prosthesis. In general, a basic concept of visual prosthesis is electrically stimulating nerve tissues associated with vision (such as the retina) to help transmit electrical signals with visual information to the brain through intact neural networks.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a visual prosthetic system including a computer system, and a wearable spectacle, the wearable spectacle linked to the computer system and including a pair of headphones, a microphone, a depth camera, a sensor, a fish-eye camera and 3D spectacle frame.

In another aspect, the invention features a visual prosthetic system including a computer system, and a wearable spectacle, the wearable spectacle linked to the computer system and comprising a pair of headphones, a microphone, a depth camera, a sensor, a fish-eye camera and 3D spectacle frame, the computer system configured to receive outputs from the depth camera, the sensor and the fish-eye camera to track a user's hand and a target object simultaneously.

In still another aspect, the invention features a visual prosthetic system including a computer system, and a wearable spectacle, the wearable spectacle linked to the computer system and including a pair of headphones, a microphone, a depth camera, a sensor, a fish-eye camera and 3D spectacle frame, the computer system configured to receive outputs from the depth camera, the sensor and the fish-eye camera to detect movement and activate an obstacle detection and warning system when a user moves and deactivate when the user stops moving.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 is a block diagram.

FIG. 2 is an architectural diagram.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

The present invention is an intelligent visual prosthesis system and method. The present invention enables detection, recognition, and localization of objects in three dimensions (3D). Core functions are based on deep neural network learning. The neural network architecture that we use is able to classify thousands of objects and, combined with information from a depth camera, localize the objects in three dimensions.

The present invention provides a small but powerful wearable prosthesis. Deep learning requires a powerful graphics processing unit (GPU) and, until recently, this would have required a desktop or large laptop computer. However, our system is a minimally conspicuous wearable device, such as, for example, a smartphone. In one implementation, this present invention uses a NVIDIA® based computer, which is about the size of a computer mouse. This low power quad core computer is specifically designed for GPU-intensive computer vision and deep learning and runs on a rechargeable battery pack. We also use very small range finding camera that provides depth mapping to complement two dimensional (2D) information from a red, green, blue (RGB) camera.

The present invention uses a twofold approach to object recognition. First, the presence of certain classes of objects are always announced via headphones (Automatic Mode). These include objects the user wants automatically announced such as obstacles and hazards as well as people. Second, with a small wearable microphone the user can manually query the device (Query Mode). By voice instruction, the user can have the system indicate if an object is present and, if so, where it is. Examples are a cell phone, a utensil dropped on the floor, or a can of soup on the shelf.

The type of auditory information provided to the user depends on the user's intent. At the most basic, the user can request a summary of the objects recognized by the RGB camera (e.g., two people, table, cups, and so forth). The user can also request information in “recognize and localize mode.” In this case, the user asks the system if a particular object is present and, if so, the system announces the location of the object using 3D sound rendering so that the announcement of the object appears to come from the object's direction. This is appropriate for situations in which the user would like to know what is in their vicinity, but he/she does not intend to physically interact with the object in a precise manner. In “grasp mode” the system gives the user auditory cues to move their hand based on proximity of an object to the hand. This latter mode facilitates grasping and using objects. Finally, if the person wants to navigate toward an object (door, store checkout, and so forth) the system indicates the object's location and warns the user of the locations of obstacles that are approached in their path as they walk.

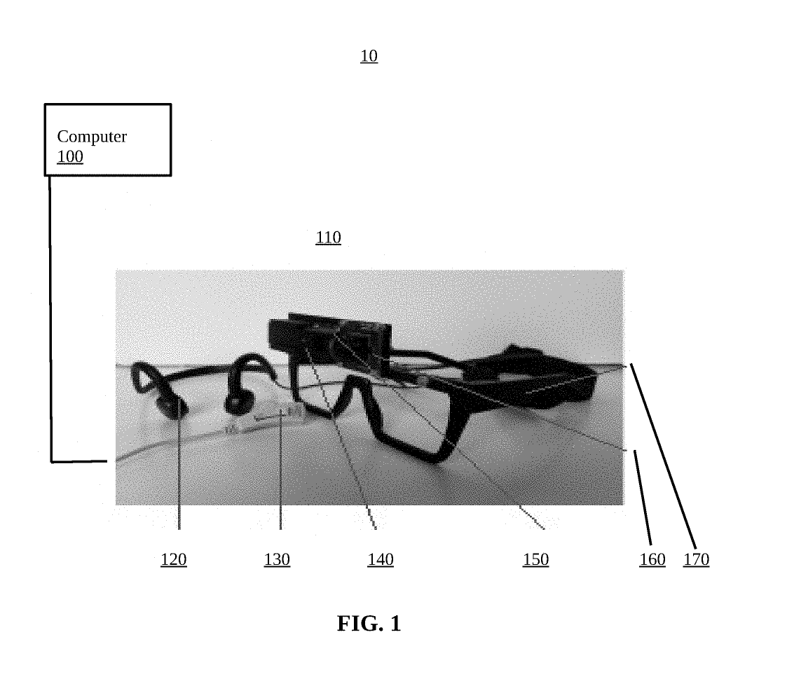

The prosthetic system of the present invention includes data input devices, processors, and outputs. In FIG. 1, an exemplary visual prosthetic system 10 includes a computer system 100 linked to spectacle 110. The spectacle 110 includes headphones 120, microphone 130, depth camera 140, sensor 150, fish-eye camera 160 and 3D spectacle frame 170. The sensor 150 is located behind the camera 140 and includes at least a magnetometer, a gyroscope and an accelerometer. Input from the RGB camera is the basis for most object recognition functions (exceptions include obstacles, stairs and curbs which are more easily detected through depth mapping). The depth camera maps the distances of objects identified by the RGB camera. Taken together, information from the two cameras establishes the 3D locations of objects in the environment and the orientation sensor links camera measurements across time. Information is conveyed to the user through bone conduction headphones (e.g., Aftershokz AS450) with speakers that sit in front of the ears, so as not to interfere with normal hearing. The headphones incorporate a microphone that accepts voice commands to locate particular objects. System software runs on a microcomputer worn on a belt with a rechargeable battery.

In a preferred embodiment, the software used is the YOLO 9000 convolutional neural network (CNN) to implement deep learning for real-time object classification and localization. The deep learning system gives pixel coordinates for detected objects (e.g., 200 pixels right, 100 pixels down). We convert these coordinates to angle coordinates relative to the camera (e.g., 30 degrees to the right, 10 degrees up). However, the fish-eye camera has significant distortion. We compensate for this by calibrating the camera using a linear regression model on labeled data.

This CNN has nineteen convolutional layers and five pooling layers; it can presently classify 9000 object categories such as people, household objects (e.g., chair, toilet, hair drier, cell phone, computer, toaster, backpack, handbag, and so forth) and outdoor objects (e.g., bicycle, motorcycle, car, truck, boat, bus, train, fire hydrant, traffic light, and so forth). As objects do not generally appear and disappear rapidly from a person's field of view, it would be computationally wasteful to run recognition and localization at a high frame rate. To keep the present system updated about object locations as the user moves their head, head movements are tracked with the orientation sensor that runs at a high frame rate. The orientation sensor communicates with the computer using the I²C serial protocol. Based on output from the cameras and orientation sensor, a 3D sound renderer (e.g., implemented in OpenAL), based on a head-related transfer function, is used to announce the 3D locations of objects through the bone conduction headphones.

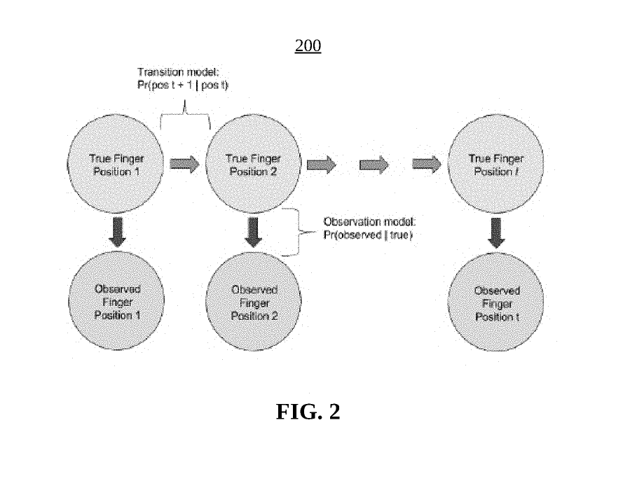

As shown in FIG. 2, an automatic process and a query process make use of the object recognition and localization output. The automatic process recognizes and locates items the user would like automatically announced. The query process enables the user to give a voice-initiated command to locate an object of interest.

More specifically, the automatic process runs continuously using the deep learning results to identify objects the user wishes to always be informed of. An example is the coming or going of people from the area within the RGB camera's wide field of view. Obstacles are always announced if they exceed a size threshold, are within a distance threshold, and are approaching the user. The automatic process is important for navigation, detecting hazards, and keeping the user updated about people in their vicinity.

The automatic process is complemented by the query process that enables the user to locate objects of interest. The object could be food in a pantry, items on a store shelf, a door in an office building, or an object dropped on the floor. To accomplish these tasks, the system accepts a voice command and the CNN locates the object in 3D based on input from the sensors. In one implementation, speech recognition uses the open source Pocketsphinx software (Carnegie Mellon). Speech recognition comes in two forms, keyword detection and recognition from a large vocabulary. While both have merits, we are using a large vocabulary for our device to differentiate between the names of detected objects. Our system can pick up certain key words very well, even distinguishing homophones. The query process is valuable for locating objects, setting targets to navigate toward, and initiating grasp mode.

In an embodiment, the auditory information the user receives is implemented using the cross-platform OpenAL SDK and the SOFT toolbox for 3D audio. Auditory information is delivered in different modes depending on the user's behavioral goal. In all functional modes, the first step is for the CNN to detect a desired object using input from the RGB camera. In some cases, input from the depth camera is also used to locate objects in 3D. The OpenAL functions are then used to make an auditory identifier of the object emanate from the object location. Accurate estimates of azimuth and elevation can be made if sounds are presented to subjects using their individual head related transfer function (HRTF). Given the complexity and expense of measuring each individual's HRTF, in a preferred embodiment the system uses generic HRTFs that have been shown to give good localization. The HRTF manipulates the interaural delay, interaural amplitude, and frequency spectrum of the sound to render the 3D spatial location of an object and deliver it to the user through the binaural bone-conduction headphones. In recognize-and-localize mode the system output is the object identifier spoken such that it appears to come from the object location.

In hand tracking/grasp mode, the user wants to interact with objects rather than a person, chair, computer, and cell simply noting their location, and the audio output requirements are different. How do we locate the user's hand? First, we attempt to segment the user's arm using a depth camera. We initially locate a pixel on the arm by assuming that it is the closest object to the camera. Then, we trace the arm until reaching the hand by finding all pixels that are “connected” to the original arm pixel. As shown in FIG. 2, to improve accuracy, we add a temporal smoothing algorithm using a Hidden Markov Model 200.

In one embodiment, the system 10 tracks the user's hand and a target object simultaneously, and guides the user's hand to grasp the target object using sound cues. Sound cues for “hand guidance” may include, for example, verbal directional cues (e.g., “Right,”, “left a little,” “forward”), hand-relative 3D sound cues, or the use of sounds with varying pitch, timbre, volume, repetition frequency, low-frequency oscillation, or other sound properties to indicate the position of a target object relative to the user's hand.

In another embodiment, the system 10 tracks the user's hand and a target object simultaneously, and guides the user's hand to grasp the target object using 3D sound cues (also referred to as “spatialized sound,” “virtual sound sources,” and “head related transfer function”) to indicate the position of an object relative to the user's hand. Here, the sounds are played in a non-conventional coordinate system relative to the position of the user's hand, rather than relative to the head.

System 10 is a wearable device that automatically detects when the user is walking, activates an obstacle detection and warning system when the user begins walking, and deactivates when the user stops walking.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A visual prosthetic system comprising:
a computer system; and
a wearable spectacle, the wearable spectacle linked to the computer system and comprising a pair of headphones, a microphone, a depth camera, a sensor, a fish-eye camera and 3D spectacle frame.

2. The visual prosthetic system of claim 1 wherein the sensor is located behind the camera.

3. The visual prosthetic system of claim 2 wherein the sensor includes at least a magnetometer, a gyroscope and an accelerometer.

4. The visual prosthetic system of claim 1 wherein the computer system comprises a 3D sound renderer that announces 3D locations of objects through the pair of headphones based on output from the depth camera, the sensor and the fish-eye camera.

5. The visual prosthetic system of claim 4 wherein the computer system further comprises:
an automatic process configured to recognize and locate items a user would like automatically announced.

6. The visual prosthetic system of claim 5 wherein the computer system further comprises:
a query process configured to enable the user to give a voice-initiated command to locate an object of interest.

7. The visual prosthetic system of claim 5 wherein the computer system further comprises:
a speech recognition engine.

8. A visual prosthetic system comprising:
a computer system; and
a wearable spectacle, the wearable spectacle linked to the computer system and comprising a pair of headphones, a microphone, a depth camera, a sensor, a fish-eye camera and 3D spectacle frame, the computer system configured to receive outputs from the depth camera, the sensor and the fish-eye camera to track a user's hand and a target object simultaneously.

9. The visual prosthetic system of claim 8 wherein the computer system is further configured to guide the user's hand to grasp the target object using sound cues.

10. The visual prosthetic system of claim 9 wherein the sound clues are selected from the group consisting of verbal directional cues, hand-relative 3D sound cues, and sounds with varying pitch, timbre, volume, repetition frequency, low-frequency oscillation, or other sound properties.

11. The visual prosthetic system of claim 10 wherein the 3D sound cues comprise sounds played in a non-conventional coordinate system relative to the position of the user's hand.

12. A visual prosthetic system comprising:
a computer system; and
a wearable spectacle, the wearable spectacle linked to the computer system and comprising a pair of headphones, a microphone, a depth camera, a sensor, a fish-eye camera and 3D spectacle frame, the computer system configured to receive outputs from the depth camera, the sensor and the fish-eye camera to detect movement and activate an obstacle detection and warning system when a user moves and deactivate when the user stops moving.

13. The visual prosthetic system of claim 12 wherein the sensor includes at least a magnetometer, a gyroscope and an accelerometer.

14. The visual prosthetic system of claim 12 wherein the computer system comprises a 3D sound renderer that announces 3D locations of objects through the pair of headphones based on output from the depth camera, the sensor and the fish-eye camera.

15. The visual prosthetic system of claim 12 wherein the computer system comprises a speech recognition engine.
